SEQUENCE LISTING (19 sequences)

SEQ ID NO: 1 - RNA, 22 nt, synthetic construct
  source 1..22: mol_type = other RNA
  ncRNA 1..22: ncRNA_class = miRNA
cagcagcaattcatgttttgaa

SEQ ID NO: 2 - RNA, 21 nt, synthetic construct
  source 1..21: mol_type = other RNA
  ncRNA 1..21: ncRNA_class = miRNA
caaaacgtgaggcgctgctat

SEQ ID NO: 3 - RNA, 23 nt, synthetic construct
  source 1..23: mol_type = other RNA
  ncRNA 1..23: ncRNA_class = miRNA
tagcagcgggaacagttctgcag

SEQ ID NO: 4 - RNA, 23 nt, synthetic construct
  source 1..23: mol_type = other RNA
  ncRNA 1..23: ncRNA_class = miRNA
ggggtattgtttccgctgccagg

SEQ ID NO: 5 - RNA, 23 nt, synthetic construct
  source 1..23: mol_type = other RNA
  ncRNA 1..23: ncRNA_class = miRNA
tcggggatcatcatgtcacgaga

SEQ ID NO: 6 - RNA, 22 nt, synthetic construct
  source 1..22: mol_type = other RNA
  ncRNA 1..22: ncRNA_class = miRNA
tgtgacagattgataactgaaa

SEQ ID NO: 7 - RNA, 22 nt, synthetic construct
  source 1..22: mol_type = other RNA
  ncRNA 1..22: ncRNA_class = miRNA
ttttgcgatgtgttcctaatat

SEQ ID NO: 8 - RNA, 22 nt, synthetic construct
  source 1..22: mol_type = other RNA
  ncRNA 1..22: ncRNA_class = miRNA
attgggaacattttgcatgtat

SEQ ID NO: 9 - RNA, 22 nt, synthetic construct
  source 1..22: mol_type = other RNA
  ncRNA 1..22: ncRNA_class = miRNA
ttttgcgatgtgttcctaatat

SEQ ID NO: 10 - RNA, 22 nt, synthetic construct
  source 1..22: mol_type = other RNA
  ncRNA 1..22: ncRNA_class = miRNA
attggggacattttgcattcat

SEQ ID NO: 11 - RNA, 22 nt, synthetic construct
  source 1..22: mol_type = other RNA
  ncRNA 1..22: ncRNA_class = miRNA
ttttgcaatatgttcctgaata

SEQ ID NO: 12 - RNA, 22 nt, synthetic construct
  source 1..22: mol_type = other RNA
  ncRNA 1..22: ncRNA_class = miRNA
ttgggatcattttgcatccata

SEQ ID NO: 13 - RNA, 98 nt, synthetic construct
  source 1..98: mol_type = other RNA
  ncRNA 1..98: ncRNA_class = miRNA
cgaggggatacagcagcaattcatgttttgaagtgttctaaatggttcaaaacgtgaggcgctgctataccccctcgtggggaaggtagaaggtgggg

SEQ ID NO: 14 - RNA, 71 nt, synthetic construct
  source 1..71: mol_type = other RNA
  ncRNA 1..71: ncRNA_class = miRNA
tgccctagcagcgggaacagttctgcagtgagcgatcggtgctctggggtattgtttccgctgccagggta

SEQ ID NO: 15 - RNA, 97 nt, synthetic construct
  source 1..97: mol_type = other RNA
  ncRNA 1..97: ncRNA_class = miRNA
cagatctcagacatctcggggatcatcatgtcacgagataccagtgtgcacttgtgacagattgataactgaaaggtctgggagccactcatcttca

SEQ ID NO: 16 - RNA, 91 nt, synthetic construct
  source 1..91: mol_type = other RNA
  ncRNA 1..91: ncRNA_class = miRNA
aaacgatactaaactgtttttgcgatgtgttcctaatatgcactataaatatattgggaacattttgcatgtatagttttgtatcaatata

SEQ ID NO: 17 - RNA, 100 nt, synthetic construct
  source 1..100: mol_type = other RNA
  ncRNA 1..100: ncRNA_class = miRNA
ccaaagaaagatgctaaactatttttgcgatgtgttcctaatatgtaatataaatgtattggggacattttgcattcatagttttgtatcaataatatgg

SEQ ID NO: 18 - RNA, 78 nt, synthetic construct
  source 1..78: mol_type = other RNA
  ncRNA 1..78: ncRNA_class = miRNA
gcagaattatttttgcaatatgttcctgaatatgtaatataagtgtattgggatcattttgcatccatagttttgtat

SEQ ID NO: 19 - RNA, 786 nt, Homo sapiens
  source 1..786: mol_type = other RNA; note = Homo sapiens MIR503 host gene (MIR503HG)
  ncRNA 1..786: ncRNA_class = miRNA
gaaggtagaaggtggggtctgccggacgcgtgttcctgccaccaggtgcccgctccccgcgaggccggctcaggagcagaaggaagcccggtgccagccagccttcctgaaagaccaagcccgcgccatccggcttcctccagtggacgcctgcaggacccaggaatgtttttcttgaaggcatccagcatctccagttagcagtactgattttttttccccccaacaaaggaacactacatcaacactgttggcggggacctggacacagaagactcctgtttcaagaaaatacaatcatctctcaaaggctgtaatttatatgcattttaaaactctaggcattgaaaaccacccaagtgtcccaaatagaagggtaatatataatcaatcactcagtgtaatattatacatcctttaaaaatgttattgggaaatgttttatgatctgtaagtccaaggaatcctctcccaccatttctttccccccgctgttcccccatacccacacttctttgttccaattggcatgtaaatttggttttcccgccaaatgagtcagtcatgatgggaacctcaactgatttgaacagatgtgtgtcaatgttacttggaaaactagatgtcaataaccagggtcacagaaaaaggcagtggtcacaactctgtaaaaatgtatgcatgcacacagacaagaactaaagtggaaccccacacaggaaaacagtgggctgtactccagtgctgggacattgaatgactgtatgctgcttttgatttccgtta

(19) United States
(12) Patent Application Publication
(10) Pub. No.: US 2023/0304007 A1
(43) Pub. Date: Sep. 28, 2023

(54) COMPOSITIONS AND METHODS OF TREATMENT OF MUSCLE DISORDERS BY TARGETING H19X-ENCODED NON-CODING RNAS

(71) Applicant: University of Houston System, Houston, TX (US)

(72) Inventors: Yu Liu, Houston, TX (US); Robert J. Schwartz, Houston, TX (US); Xiaopeng Shen, Houston, TX (US); Rui Liang, Houston, TX (US); Shreesti Shrestha, Houston, TX (US)

(21) Appl. No.: 18/190,097

(22) Filed: Mar. 26, 2023

Related U.S. Application Data
(60) Provisional application No. 63/269,975, filed on Mar. 26, 2022.

(51) Int. Cl. C12N 15/113; C12N 9/22; A61P 21/00

(52) U.S. Cl. CPC C12N 15/113; C12N 9/22; A61P 21/00; C12N 2310/141

(57) ABSTRACT

Provided here are compositions and methods of preventing or treating a muscle disorder in a subject, such as muscle wasting, cachexia, sarcopenia and heart failure. The compositions include inhibitors targeting one or more of the H19X-encoded non-coding RNAs. The inhibitors can inhibit expression of one or more of the H19X-encoded non-coding RNAs or inhibit interaction between one or more of the H19X-encoded non-coding RNAs and their natural target mRNA. Also, provided are methods of preventing or treating a muscle disorder in a subject by inducing specific mutations to decrease levels of one or more of the H19X-encoded non-coding RNAs utilizing gene editing tools such as, but not limited to, integrases, CRISPR/Cas nucleases, TALAN nucleases, zinc finger Nucleases, triplex forming oligonucleotides, or combinations thereof.

### GOVERNMENT SUPPORT

This invention was made with government support under grant no. PR162075 awarded by Department of Defense's Congressionally Directed Medical Research Programs. The government has certain rights in the invention.

### TECHNICAL FIELD

The disclosure relates to compositions and methods of treatment of muscle disorders by targeting H19X-encoded non-coding RNAs.

### BACKGROUND

Loss of skeletal muscle mass (i.e. muscle atrophy/wasting) is closely associated with poor prognosis in many chronic diseases, including myopathies, muscular dystrophies, cancers, diabetes, sepsis, and heart failure. Muscle loss also contributes to decreased quality of life in aging. Muscle atrophy results from a reduced rate of protein synthesis, accelerated protein degradation, or both. The ubiquitin—proteasome system and autophagy are two major proteolytic mechanisms that are activated in skeletal muscle in various atrophying conditions. The activity of ubiquitin—proteasome system and autophagy is regulated through coordinated activation of several signaling pathways, such as nuclear factor-κB and p38 mitogen-activated protein kinase pathways. Moreover, the canonical transforming growth factor-β family members, such as myostatin and activin that function through activin receptors and activation of SMAD2/3 transcription factors, are important drivers of muscle wasting. In contrast, the IGF1/AKT/mTOR pathway increases the rate of protein synthesis leading to skeletal muscle hypertrophy. Activation of this pathway also inhibits muscle protein degradation through distinct mechanisms. For example, gene expression of two muscle-specific E3 ubiquitin ligases, MuRF1 and MAFbx/Atrogin-1, is regulated by transcription factors FoxO1/3, which are removed from the nucleus upon phosphorylation by AKT. The transforming growth factor-β inhibits AKT activity through stimulating SMAD signaling.

MicroRNAs (miRNAs) are small non-coding RNAs that regulate post-transcriptional gene expression. The abundance of some miRNAs is altered in wasting human muscle biopsies. Eight miRNAs were differentially expressed in rectus abdominis of cachectic and non-cachectic cancer patients. Thirty-two differentially expressed miRNAs were identified in the quadriceps of chronic obstructive pulmonary disease patients with a low fat-free mass index: 26 were down-regulated, while 6 were up-regulated. Remarkably, five of the six up-regulated miRNAs were encoded by the H19X locus located on Xq26.3. H19X-encoded miRNAs are differentially expressed in skeletal muscle in several cachectic/wasting diseases. The H19X-encoded miRNAs-322(424)/miR-503 cluster was highly expressed in bipotent cardiac and skeletal muscle progenitors. During embryonic stem cell differentiation, the cluster plays an essential role in cardiomyocyte specification by targeting and inhibiting neuroectoderm lineage factors. There is a need for therapeutic agents targeting specific portions of the H19X-encoded genes to treat muscle disorders.

### SUMMARY

Applicant has recognized the shortcomings in the art and the need for therapeutic agents targeting specific portions of the H19X-encoded genes to treat muscle disorders. H19X-encodes a number of non-coding RNAs including miR-424, miR-503, miR503HG, miR-542, miR-450a1, miR-450a2 and miR-450b that are specifically expressed in embryonic heart and muscles. They are not normally express in adult tissues, but they are activated in muscle wasting associated with cancer and chronic diseases, in sarcopenia (age-related muscle wasting), and in heart failure.

Embodiments include methods of preventing or treating a muscle disorder in a subject in need thereof by administering an inhibitor of one or more of miR-424, miR-503, miR503HG, miR-542, miR450a1, miR-450a2, and miR-450b. The muscle disorders include muscle wasting, cachexia, sarcopenia, and heart failure. Heart failure can result from cardiomyopathy. The inhibitor can reduce the expression of the miRNA or affect the activity of the miRNA. In certain embodiments, the inhibitor reduces expression of one or more of miR-424, miR-503, miR503HG, miR-542, miR450a1, miR-450a2, and miR-450b. The inhibitor can be a nucleic acid selected from a group consisting of a DNA, an RNA, an antagomir, a siRNA, a shRNA, and an oligonucleotide. The equivalent of miR-424 in humans is miR-322 miRNA in mice.

In certain embodiments, the inhibitor affects activity of one or more of miR-424, miR-503, miR503HG, miR-542, miR450a1, miR-450a2, and miR-450b. The inhibitor can act by reducing interaction between one or more of miR-424, miR-503, miR503HG, miR-542, miR450a1, miR-450a2, and miR-450b and their natural target mRNA. The inhibitor can be a nucleic acid selected from a group consisting of a DNA, an RNA, an antagomir, a siRNA, a shRNA and an oligonucleotide. Specific antisense-based inhibitors of one or more of miR-424, miR-503, miR503HG, miR-542, miR450a1, miR-450a2, and miR-450b preserve muscle in muscular disorders, such as muscle wasting, cachexia, sarcopenia, and cardiomyopathy.

The inhibitor can be a nucleic acid binding to all or a portion of a base sequence of one or more of miR-424, miR-503, miR503HG, miR-542, miR450a1, miR-450a2, and miR-450b. In an embodiment, the sequence of one or more of miR-424, miR-503, miR503HG, miR-542, miR450a1, miR-450a2, and miR-450b is derived from a human, and includes not only a mature sequence but also a precursor sequence. The nucleic acid inhibitor can be an antisense oligonucleotide comprising a sequence partially or completely complementary to the base sequence of one or more of miR-424, miR-503, miR503HG, miR-542, miR450a1, miR-450a2, and miR-450b.

Certain embodiments include methods of preventing or treating a muscle disorder in a subject in need thereof by inducing a mutation to reduce expression of one or more of miR-424, miR-503, miR503HG, miR-542, miR450a1, miR-450a2, and miR-450b. In some embodiments, the mutation is an insertion mutation to block transcription of one or more of miR-424, miR-503, miR503HG, miR-542, miR450a1, miR-450a2, and miR-450b. Certain methods can include inducing a mutation by editing a sequence of one or more of miR-424, miR-503, miR503HG, miR-542, miR450a1, miR-450a2, and miR-450b by a nuclease. The nuclease can be one or more of a meganuclease, a zinc-finger nuclease, a transcription activator-like effector nuclease, and a CRISPR/Cas nuclease.

Embodiments of therapeutic agents targeting specific portions of the H19X-encoded genes to treat muscle disorders include antagomirs, RNA-sponges, CRISPR-mediated promoter silencing or gene editing, and other agents targeting H19X non-coding RNAs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation in the figures of the accompanying drawings.

FIGS. 1A-1M illustrate that skeletal muscle-specific overexpression of miR-322/-503 impeded muscle growth. FIG. 1A is a diagrammatic representation of the transgenic gene expression cassette. The −6.5 kb MCK promoter was used to drive miR-322/-503 expression. FIG. 1B is a set of graphical representations of the levels of expression of miR-322 (left panel) and miR-503 (right panel) in skeletal muscles of the wild-type (wt) and transgenic animals (tg) at birth (n=6 vs. 5). FIG. 1C is a graphical representation of the body weight of the wild-type and transgenic animals. FIG. 1D is a graphical representation of the body weight of the wild-type and transgenic animals at 4 months of age (n=6 vs. 5). FIG. 1E is a graphical representation of the grip strength of the wild-type and transgenic animals. FIG. 1F is a set of photographs of the transgenic and wild-type animals with back and hind limb muscles exposed. FIG. 1G is a photograph of the hind limb muscles from transgenic and wild-type animals. Quad refers to quadriceps, TA refers to tibialis anterior, Sol refers to soleus, EDL refers to extensor digitorum longus, and GA refers to gastrocnemius muscles. FIG. 1H is a graphical representation of the masses of gastrocnemius muscles of the wild-type and transgenic animals. FIG. 1I is a graphical representation of the tibia bone lengths of the wild-type and transgenic animals. FIG. 1J is a graphical representation of the mean cross-sectional area (CSA) of TA muscle of the wild-type and transgenic animals (n=5 vs. 5). FIG. 1K is a graphical representation of the frequency histograms of CSA in TA muscles of the wild-type and transgenic animals (n=5 vs. 5). FIG. 1L is a set of photographs of the succinate dehydrogenase (SDH) activity staining of the wild-type and transgenic animals. FIG. 1M is a graphical representation of the oxidative fiber percentage based on SDH staining of the wild-type and transgenic animals (n=10 vs. 7). For all panels, *, p<0.05; **, p<0.01; ****, p<0.0001.

FIGS. 2A-2L illustrate that inactivation of the H19X locus promoted muscle growth and rendered resistance to starvation-induced atrophy. FIG. 2A is a diagrammatic representation of the transgenic gene expression cassette with the insertion mutation that blocks downstream transcription in the H19X locus. FIG. 2B is a set of graphical representations of the levels of expression of miR-322 (left panel), miR-503 (middle panel), and miR-542 (right panel) in mutant vs. wild-type muscles (n=4 vs. 6). FIG. 2C is a graphical representation of the body weight of the wild-type and mutant animals at 6 months old. FIG. 2D is a set of graphical representations of the hind limb muscle masses of the wild-type and mutant animals, specifically TA (left panel), GA (middle panel), and Quad muscles (right panel). FIG. 2E is a graphical representation of the frequency histograms of CSA in the wild-type and mutant muscles (n=5 vs. 5). FIG. 2F is a set of photographs of the SDH activity staining of the wild-type and mutant animals. FIG. 2G is a graphical representation of the percentage of oxidative fibers in mutant animals and wild-type animals as determined by SDH staining (n=11 vs. 11). FIG. 2H is a set of graphical representations of the levels of expression of eIF4E (left panel), eIF4B (middle panel), and eIF3M (right panel) expression in mutant vs. wild-type muscles (n=4 vs. 6). FIG. 2I is a set of photographs of the Western Blot analysis of the new protein synthesis in mutant vs. wild-type muscles. FIG. 2J is a graphical representation of the new protein synthesis in mutant vs. wild-type muscles, which was upregulated in mutant muscles. FIG. 2K is a graphical representation of the body weight loss in mutant vs. wild-type animals after 48 hours of starvation. FIG. 2L is a set of graphical representations of the relative weight of sol muscles (left panel), TA muscles (middle panel), and GA muscles (right panel) in mutant vs. wild-type animals after 48 hours of starvation. Interruption of the H19X locus partially rescued starvation-induced muscle wasting (n=10 vs. 11). Shown are muscle-weight/bodyweight ratios of starved animals normalized to that of regularly fed wild-type littermates.

FIG. 3A is a graphical representation of the levels of miR503HG in male and female subjects. Higher levels of miR503HG are associated with female sex. FIG. 3B is a graphical representation of the levels of miR503HG among subjects in ten-year intervals of age. FIG. 3C is a graphical representation of the levels of miR503HG in male and female subjects among subjects in ten-year intervals of age. FIG. 3D is a graphical representation of the levels of miR503HG at various points on a Hardy scale (1: violent and fast death, 2: fast death of natural causes, 3: intermediate death, 4: slow death from chronic illness). Higher levels of miR503HG are associated with older age and slow death. Median level is indicated by a horizontal line. *, p<0.05; **, p<0.01; ****, p<0.001.

FIGS. 4A-4H are photographic images of levels of miR503HG in male and female subjects at the age of 60-69. High levels of miR503HG are indicative of severe human muscle atrophy.

FIGS. 5A-5F illustrate that tet-induced heart-specific expression of miR-322/503 resulted in rapid and severe dilated cardiomyopathy. FIG. 5A is a diagrammatic representation of the transgene strategy. FIG. 5B is a set of graphical representation of the heart expression of the transgene: miR-322 (left panel) and miR-503 (right panel). FIG. 5C is a set of graphical representation of the effect of ectopic miR-322/503 on the systolic pump function of the heart, specifically ejection fraction (left panel) and fractional shortening (right panel). FIG. 5D is a set of graphical representation of the expression of markers of cardiac remodeling: ANF (first panel from the left), BNP (second panel), α-MHC (third panel), and β-MHC (fourth panel). Ectopic miR-322/503 induced markers of cardiac remodeling. FIG. 5E is a set of photographic images of the heart in wild type and transgenic animals. FIG. 5F is a set of photographic images of muscles of wild type and transgenic animals stained with hematoxylin and eosin (H&E) stain (top and bottom, left panels) and Masson's Trichrome stain (top and bottom, right panels), respectively.

FIGS. 6A-6E illustrate that inhibitors against miR-322/-503 augmented myotube growth and resistance to starvation-induced atrophy. FIG. 6A is a set of photographic images of C2C12-derived myotubes treated with control, miR-322i, miR-503i, or both, under normal and starvation conditions. FIG. 6B is a graphical representation of the myotube diameter of C2C12-derived myotubes treated with control, miR-322i, miR-503i, or both, under normal (F) and starvation (S) conditions, showing that both miR-322i and miR-503i upregulated myotube growth and partially protected them from starvation-induced wasting. FIG. 6C is a photographic image showing expression of eIF4G1, eIF4B, eIF2B5, eIF3M, and eIF4E C2C12-derived myotubes treated with control, miR-322i, miR-503i, or both, under normal and starvation conditions. Expression of eIF4G1, eIF4B, eIF2B5 and eIF3M proteins, but not eIF4E, were boosted by miR-322i/-503i. FIG. 6D is a photographic image showing expression of puro-tagged peptides in C2C12-derived myotubes treated with control, miR-322i, miR-503i, or both, under normal and starvation conditions. FIG. 6E is a graphical representation of the expression levels of puro-tagged peptides in C2C12-derived myotubes treated with control, miR-322i, miR-503i, or both, under normal and starvation conditions. Impaired protein synthesis in starved myotubes was partially rescued by miR-322i/-503i. For all panels, *, p<0.05; **, p<0.01; ****, p<0.0001, and ns, not significant.

FIGS. 7A-7D demonstrate that expression of miR-322/miR-503 is inversely related to postnatal skeletal muscle growth. FIG. 7A is a photographic image of the whole-mount β-gal staining of embryonic organs at E17.5. FIG. 7B is a graphical representation of the expression levels of miR-322 (upper panel) and miR-503 (lower panel) expression in both tongue and hindlimb muscles. FIG. 7C is a graphical representation of the percentage increase in mass of Sol (left panel), TA (middle panel), and GA (right panel) muscles as compared to the body weight in control mice and mice subject to four weeks of mandatory treadmill training. FIG. 7D is a graphical representation of the expression levels of miR-322 (left panel), miR-503 (middle panel), and miR-542 (right panel) expression in control mice and mice subject to four weeks of mandatory treadmill training.

### DETAILED DESCRIPTION

Muscle wasting and cachexia are complications of many diseases, including but not limited to cancer, sepsis, chronic obstructive pulmonary diseases, neurological disorders, and arthritis. Muscle wasting is also related to aging. Muscle wasting and cachexia increase the rates of fatality in the underlying diseases and they severely diminish life quality. There are no cures for muscle wasting, cachexia, and sarcopenia. Patients are treated by nutrition support and exercises, which are not effective for most patients. Heart failure is among the leading causes of death. Treatment is usually targeted towards reducing the pre- and after-load of the heart. Despite the many drug options, patients still have one of the worst 5-year survival rates among life-threatening diseases.

As disclosed herein, H19X non-coding RNAs are responsible for skeletal muscle wasting and heart failure. Embodiments include compositions and methods of targeting these non-coding RNAs in treating diseases, such as muscle wasting, cachexia, sarcopenia, and heart failure. Certain H19X-encoded non-coding RNAs of interest include one long non-coding RNA (lncRNA) miR503HG and 7 miRNAs—miR-424, miR-503, miR-542, miR450a1, miR-450a2, and miR-450b.

Embodiments of the present disclosure relates to therapeutic agents targeting one or more of these lncRNAs of interest, such as miR503HG, for preventing or treating muscle wasting. Embodiments also include methods for screening such an agent, which include steps of measuring the expression level of one or more of these lncRNAs of interest, such as miR503HG, and identifying an agent that affects the expression level of one or more of these lncRNAs of interest, such as miR503HG. Embodiments also include pharmaceutical compositions containing an inhibitor of one or more of these lncRNAs of interest, such as miR503HG, for preventing or treating a disease characterized by muscle wasting.

Embodiments of the present disclosure relates to therapeutic agents targeting one or more of these miRNAs of interest—miR-424, miR-503, miR-542, miR450a1, miR-450a2, and miR-450b for preventing or treating muscle wasting. Embodiments also include methods for screening such an agent, which include steps of measuring the expression level of one or more of these miRNAs of interest—miR-424, miR-503, miR-542, miR450a1, miR-450a2, and miR-450b, and identifying an agent that affects the expression level of one or more of these miRNAs of interest. Embodiments also include pharmaceutical compositions containing an inhibitor of one or more of these miRNAs of interest—miR-424, miR-503, miR-542, miR450a1, miR-450a2, and miR-450b for preventing or treating a disease characterized by muscle wasting.

In the present disclosure, the ‘miR’ or ‘microRNA (miRNA)’ refers to a naturally occurring single-stranded, non-coding small RNA molecule that controls gene expression by binding to complementary sequences in its target mRNAs, thereby inhibiting translation or inducing mRNA degradation.

The term “complementary” as used herein refers to an antisense oligonucleotide that is sufficiently complementary to the miRNA target under predetermined hybridization conditions or annealing conditions, specifically under physiological conditions, such that it can selectively hybridize to the target, and encompasses both partially or substantially complementary and completely complementary sequences. Substantially complementary means that, although not completely complementary, it has complementarity sufficient to bind to the target sequence and interfere with the activity of the miRNA. The term “nucleic acid” includes an oligonucleotide, a DNA, an RNA, a polynucleotide, and analogs and derivatives thereof. For example, a PNA or a mixture thereof is included.

In the present disclosure, a miRNA inhibitor can inhibit the expression of the specific miRNA or a lncRNA. In certain embodiments, it can inhibit the interaction between miRNA and its lncRNA target. In certain embodiments, it can inhibit the interaction between miRNA and its mRNA target. For example, the mRNA target for miR-424/miR-503 includes the 3′-untranslated region of translation initiation factors, such as eIF4E1, eIF4G1, eIF4B, eIF2B5, eIF3M, IGF1R, BCL2, and CDC25A. The miRNA targets for miR-542, miR-450a1, miR-450a2 and miR-450b include, but are not limited to, SMAD7, SMURF1, RP 523, TIMMDC1, MT-ND2, ACO2, and ATP5B. In the present disclosure, the miRNA inhibitor may inhibit or interfere with the action or function of miRNA or lncRNA in cells. Inhibitors can directly inhibit the binding of the miRNA to its target mRNA. In certain embodiments, these nucleic acid inhibitors can be selected from a group consisting of DNA, RNA, an antagomir (antisense oligonucleotide of miRNA), a RNA sponge, a siRNA, a shRNA and an oligonucleotide. In other embodiments, the miRNA inhibitor includes any agent capable of reducing the expression and/or activity of miRNA. The agent can include, without any limitations, a low-molecular-weight compound, an antagomir, an antisense molecule, a small hairpin RNA (shRNA) molecule, a small interfering RNA (siRNA) molecule, a seed target LNA (locked nucleic acid) oligonucleotide, a decoy oligonucleotide, an aptamer, a ribozyme, or an antibody that recognizes a DNA:RNA hybrid. The miRNA inhibitor can be an antisense oligonucleotide which can inhibit the activity of miRNA by complementarily binding to all or a part of the precursor and/or mature sequence of the miRNA.

An antagomir is a chemically modified single-stranded oligonucleotide and is used to silence one or more of these miRNAs of interest—miR-424, miR-503, miR503HG, miR-542, miR450a1, miR-450a2, and miR-450b. The antagomir has a sequence which is at least partially or completely complementary to one or more of these miRNAs of interest—miR-424, miR-503, miR-542, miR450a1, miR-450a2, and miR-450b. The antagomir can include one or more modification (e.g., 2′-O-methyl-sugar modification or 3′-cholesterol modification). The antagomir can contain one or more phosphorothioate linkage and have a phosphorothioate backbone at least in part. In the present disclosure, the appropriate length of the antagomir for inhibiting the expression of miRNA is 7-50 nt (nucleotides), specifically 10-40 nt, more specifically 15-30 nt, more specifically 15-25 nt, more specifically 20-22 nt, although not being limited thereto.

A sponge RNA is a small synthetic RNA that binds to multiple miRNAs, which have the same sequence in their ‘seed region’. When the sponge is expressed at high levels, it specifically inhibits the activity of a family of miRNAs sharing a common seed (miRNA nucleotides 2-7, the major specificity determinant for target recognition).

Mature miRNA sequence with accession number, and the “seed sequence” as underlined text are provided below in Table 1.

TABLE 1

| SEQ ID | | |
|---|---|---|
| NO. | NAME | SEQUENCE |
| SEQ ID | hsa-miR-424-5p | CAGCAGCAAUUCAUGUUUUGAA |
| NO. 1 | MIMAT0001341 | |
| SEQ ID | hsa-miR-424-3p | CAAAACGUGAGGCGCUGCUAU |
| NO. 2 | MIMAT0004749 | |
| SEQ ID | hsa-miR-503-5p | UAGCAGCGGGAACAGUUCUGCAG |
| NO. 3 | MIMAT0002874 | |
| SEQ ID | hsa-miR-503-3p | GGGGUAUUGUUUCCGCUGCCAGG |
| NO. 4 | MIMAT0022925 | |
| SEQ ID | hsa-miR-542-5p | UCGGGGAUCAUCAUGUCACGAGA |
| NO. 5 | MIMAT0003340 | |
| SEQ ID | hsa-miR-542-3p | UGUGACAGAUUGAUAACUGAAA |
| NO. 6 | MIMAT0003389 | |
| SEQ ID | hsa-miR-450a-5p | UUUUGCGAUGUGUUCCUAAUAU |
| NO. 7 | MIMAT0001545 | |
| SEQ ID | hsa-miR-450a-1-3p | AUUGGGAACAUUUUGCAUGUAU |
| NO. 8 | MIMAT0022700 | |
| SEQ ID | hsa-miR-450a-5p | UUUUGCGAUGUGUUCCUAAUAU |
| NO. 9 | MIMAT0001545 | |
| SEQ ID | hsa-miR-450a-2-3p | AUUGGGGACAUUUUGCAUUCAU |
| NO. 10 | MIMAT0031074 | |
| SEQ ID | hsa-miR-450b-5p | UUUUGCAAUAUGUUCCUGAAUA |
| NO. 11 | MIMAT0004909 | |
| SEQ ID | hsa-miR-450b-3p | UUGGGAUCAUUUUGCAUCCAUA |
| NO. 12 | MIMAT0004910 | |

Pre-miRNA sequences with accession number are provided below.

SEQ ID NO. 13:
hsa-mir-424 MI0001446
CGAGGGGAUACAGCAGCAAUUCAUGUUUUGAAGUGU
UCUAAAUGGUUCAAAACGUGAGGCGCUGCUAUACCC
CCUCGUGGGGAAGGUAGAAGGUGGGG
SEQ ID NO. 14:
hsa-mir-503 MI0003188
UGCCCUAGCAGCGGGAACAGUUCUGCAGUGAGCGAU
CGGUGCUCUGGGGUAUUGUUUCCGCUGCCAGGGUA
SEQ ID NO. 15:
hsa-mir-542 MI0003686
CAGAUCUCAGACAUCUCGGGGAUCAUCAUGUCACGA
GAUACCAGUGUGCACUUGUGACAGAUUGAUAACUGA
AAGGUCUGGGAGCCACUCAUCUUCA
SEQ ID NO. 16:
hsa-mir-450a-1 MI0001652
AAACGAUACUAAACUGUUUUUGCGAUGUGUUCCUAA
UAUGCACUAUAAAUAUAUUGGGAACAUUUUGCAUGU
AUAGUUUUGUAUCAAUAUA
SEQ ID NO. 17:
hsa-mir-450a-2 MI0003187
CCAAAGAAAGAUGCUAAACUAUUUUUGCGAUGUGUU
CCUAAUAUGUAAUAUAAAUGUAUUGGGGACAUUUUG
CAUUCAUAGUUUUGUAUCAAUAAUAUGG
SEQ ID NO. 18:
hsa-mir-450b MI0005531
GCAGAAUUAUUUUUGCAAUAUGUUCCUGAAUAUGUA
AUAUAAGUGUAUUGGGAUCAUUUUGCAUCCAUAGUU
UUGUAU

Chromosome coordinates of the miRNAs are provided below.
hsa-mir-424 at chrX: 134546614-134546711 [-]
hsa-mir-503 at chrX: 134546328-134546398 [-]
hsa-mir-542 at chrX: 134541341-134541437 [-]
hsa-mir-450a-2 at chrX: 134540508-134540607 [-]
hsa-mir-450a-1 at chrX: 134540341-134540431 [-]
hsa-mir-450b at chrX: 134540185-134540262 [-]

In the present disclosure, the pharmaceutical composition may further contain, in addition to the miRNA inhibitor, one or more active ingredient exhibiting the same, similar or synergistic function for the treatment of related diseases or a compound which maintains/increases the solubility and/or absorbency of the miRNA inhibitor.

The pharmaceutical composition may further contain one or more pharmaceutically acceptable diluent, carrier and/or adjuvant in addition to the above-mentioned therapeutic agents. In addition, it can be formulated into an injectable formulation such as an aqueous solution, a suspension, an emulsion, etc., a pill, a capsule, a granule or a tablet by additionally adding a diluent, a dispersant, a surfactant, a binder and a lubricant, and it can be used by binding a target organ-specific antibody or other ligand with the carrier. The pharmaceutical composition can be formulated into a formulation for intranasal administration, intravenous administration, subcutaneous injection, intrathecal injection, inhalation administration or oral administration. The pharmaceutical composition can be prepared into a variety of unit dosage forms. The pharmaceutical composition may be administered in a pharmaceutically or therapeutically effective amount. The pharmaceutically or therapeutically effective amount means an amount sufficient to treat a disease at a reasonable benefit/risk ratio applicable to medical treatment, and an effective dose level will depend on factors including but not limited to the type and severity of the disease, the activity of the therapeutic agent, sensitivity to the therapeutic agent, the time of administration, the route of administration, the rate of excretion, and the duration of the treatment.

In addition, the pharmaceutical composition may be administered as an individual therapeutic agent or in combination with other therapeutic agents, sequentially or concurrently with conventional therapeutic agents, and may be administered either alone or in combination.

Embodiments of therapeutic regimens to treat muscle disorders include inducing specific mutations to decrease expression of one or more of miR-424, miR-503, miR503HG, miR-542, miR450a1, miR-450a2, and miR-450b. Some embodiments of the methods include inducing a mutation that is an insertion mutation to block transcription of one or more of miR-424, miR-503, miR503HG, miR-542, miR450a1, miR-450a2, and miR-450b. Mutations can be induced by utilizing gene editing tools such as, but not limited to, integrases (recombinases), clustered regularly interspaced short palindromic repeat (CRISPR) and CRISPR associated protein (Cas) nucleases, TALAN nucleases, zinc finger nucleases, triplex forming oligonucleotides, or combinations thereof. Certain methods can include inducing a mutation by editing a sequence of one or more of miR-424, miR-503, miR503HG, miR-542, miR450a1, miR-450a2, and miR-450b by a nuclease. The nuclease can be one or more of a meganuclease, a zinc-finger nuclease, a transcription activator-like effector nuclease, a CRISPR/Cas9 nuclease, a CRISPR/Cpf1 nuclease, a CRISPR/CasX nuclease, a CRISPR/CasY nuclease, and a Csm1 nuclease.

Embodiments include methods of preventing or treating a muscle wasting disorder in a subject in need thereof by administering an inhibitor of one or more of miR-424, miR-503, miR503HG, miR-542, miR450a1, miR-450a2, and miR-450b. The muscle disorders include muscle wasting, cachexia, sarcopenia and heart failure. The term “treatment” as used in the present disclosure means any action to change favorably or improve the symptoms of related diseases by administering the therapeutic agent. The term “prevention” used in the present disclosure means any action to inhibit or delay the onset of related diseases. It will be apparent to those skilled in the art that the related diseases can be prevented if the pharmaceutical composition according to the present disclosure is administered when or before early symptoms appear. Reference to miR-424, miR-503, miR503HG, miR-542, miR450a1, miR-450a2, or miR-450b includes a variant of miR-424, miR-503, miR503HG, miR-542, miR450a1, miR-450a2, or miR-450b, respectively. A variant includes a nucleic acid with at least 80% sequence similarity to a particular SEQ ID, a nucleic acid with at least 85% sequence similarity to a particular SEQ ID, a nucleic acid with at least 90% sequence similarity to a particular SEQ ID, or a nucleic acid with at least 95% sequence similarity to a particular SEQ ID.

In certain embodiments, the inhibitor reduces expression of one or more of miR-424, miR-503, miR503HG, miR-542, miR450a1, miR-450a2, and miR-450b. The inhibitor can be a nucleic acid selected from a group consisting of a DNA, an RNA, an antagomir, a RNA sponge, a siRNA, a shRNA and an oligonucleotide.

In certain embodiments, the inhibitor reduces interaction between one or more of miR-424, miR-503, miR503HG, miR-542, miR450a1, miR-450a2, and miR-450b and their natural target. For example, the target nucleic acids for miR-424/-503 code for one or more of IGF1R, BCL2, CDC25A, eIF4E, eIF4G1, eIF4B, eIF2B5 and eIF3M. For example, the target nucleic acids for miR-542 code for one or more of SMAD7, SMURF1, and RPS23. For example, the target nucleic acids for miR-450 code for one or more of TIMMDC1, MT-ND2, ACO2, and ATP5B. In certain embodiments, as the miRNAs interact with the same target, an inhibitor may play a role in reducing interactions of several miRNAs.

The inhibitor can be a nucleic acid selected from a group consisting of a DNA, an RNA, an antagomir, a siRNA, a shRNA and an oligonucleotide. Specific antisense-based inhibitors of miR-424/miR-503 preserves muscle in wasting conditions. Embodiments of therapeutic agents targeting specific portions of the H19X-encoded genes to treat muscle disorders include antagomirs, RNA-sponge, CRISPR-mediated promoter silencing and gene editing, and other agents targeting H19X non-coding RNAs.

The inhibitor can be a nucleic acid binding to all or a part of a base sequence of miR-424. In an embodiment, the sequence of miR-424 is derived from a human, and includes not only a mature sequence but also a precursor sequence, provided by SEQ ID NOS. 1, 2, or 13 or a variant thereof. The nucleic acid can be an antisense oligonucleotide containing a sequence partially or completely complementary to the base sequence of miR-424, provided by SEQ ID NOS. 1, 2, or 13 or a variant thereof.

The inhibitor can be a nucleic acid binding to all or a part of a base sequence of miR-503. In an embodiment, the sequence of this miRNA is derived from a human, and includes not only a mature sequence but also a precursor sequence, provided by SEQ ID NOS. 3, 4, or 14 or a variant thereof. The nucleic acid can be an antisense oligonucleotide containing a sequence partially or completely complementary to the base sequence of miR-503, provided by SEQ ID NOS. 3, 4, or 14 or a variant thereof.

The inhibitor can be a nucleic acid binding to all or a part of a base sequence of miR-542. In an embodiment, the sequence of this miRNA is derived from a human, and includes not only a mature sequence but also a precursor sequence, provided by SEQ ID NOS. 5, 6, or 15 or a variant thereof. The nucleic acid can be an antisense oligonucleotide comprising a sequence partially or completely complementary to the base sequence of miR-542, provided by SEQ ID NOS. 5, 6, or 15 or a variant thereof.

The inhibitor can be a nucleic acid binding to all or a part of a base sequence of miR450a1. In an embodiment, the sequence of this miRNA is derived from a human, and includes not only a mature sequence but also a precursor sequence, provided by SEQ ID NOS. 7, 8, or 16 or a variant thereof. The nucleic acid can be an antisense oligonucleotide comprising a sequence partially or completely complementary to the base sequence of miR450a1, provided by SEQ ID NOS. 7, 8, or 16 or a variant thereof.

The inhibitor can be a nucleic acid binding to all or a part of a base sequence of miR-450a2. In an embodiment, the sequence of this miRNA is derived from a human, and includes not only a mature sequence but also a precursor sequence, provided by SEQ ID NOS. 9, 10, or 17 or a variant thereof. The nucleic acid can be an antisense oligonucleotide comprising a sequence partially or completely complementary to the base sequence of miR-450a2, provided by SEQ ID NOS. 9, 10, or 17 or a variant thereof.

The inhibitor can be a nucleic acid binding to all or a part of a base sequence of miR-450b. In an embodiment, the sequence of this miRNA is derived from a human, and includes not only a mature sequence but also a precursor sequence, provided by SEQ ID NOS. 11, 12, or 18 or a variant thereof. The nucleic acid can be an antisense oligonucleotide comprising a sequence partially or completely complementary to the base sequence of miR-450b, provided by SEQ ID NOS. 11, 12, or 18 or a variant thereof.

The inhibitor can be a nucleic acid binding to all or a part of a base sequence of miR503HG. In an embodiment, the sequence of this miRNA is derived from a human, and includes not only a mature sequence but also a precursor sequence, provided by SEQ ID NO. 19 or a variant thereof. The nucleic acid can be an antisense oligonucleotide comprising a sequence partially or completely complementary to the base sequence of miR503HG, provided by SEQ ID NO. 19. For example, reference to miR503HG includes its variants, which are nucleic acids with at least 80% sequence similarity to a particular SEQ ID NO: 19, nucleic acids with at least 85% sequence similarity to SEQ ID NO: 19, nucleic acids with at least 90% sequence similarity to SEQ ID NO: 19, nucleic acids with at least 95% sequence similarity to SEQ ID NO: 19.

SEQ ID NO. 19:
GAAGGTAGAAGGTGGGGTCTGCCGGACGCGTGTTCC
TGCCACCAGGTGCCCGCTCCCCGCGAGGCCGGCTCA
GGAGCAGAAGGAAGCCCGGTGCCAGCCAGCCTTCCT
GAAAGACCAAGCCCGCGCCATCCGGCTTCCTCCAGT
GGACGCCTGCAGGACCCAGGAATGTTTTTCTTGAAG
GCATCCAGCATCTCCAGTTAGCAGTACTGATTTTTT
TTCCCCCCAACAAAGGAACACTACATCAACACTGTT
GGCGGGGACCTGGACACAGAAGACTCCTGTTTCAAG
AAAATACAATCATCTCTCAAAGGCTGTAATTTATAT
GCATTTTAAAACTCTAGGCATTGAAAACCACCCAAG
TGTCCCAAATAGAAGGGTAATATATAATCAATCACT
CAGTGTAATATTATACATCCTTTAAAAATGTTATTG
GGAAATGTTTTATGATCTGTAAGTCCAAGGAATCCT
CTCCCACCATTTCTTTCCCCCCGCTGTTCCCCCATA
CCCACACTTCTTTGTTCCAATTGGCATGTAAATTTG
GTTTTCCCGCCAAATGAGTCAGTCATGATGGGAACC
TCAACTGATTTGAACAGATGTGTGTCAATGTTACTT
GGAAAACTAGATGTCAATAACCAGGGTCACAGAAAA
AGGCAGTGGTCACAACTCTGTAAAAATGTATGCATG
CACACAGACAAGAACTAAAGTGGAACCCCACACAGG
AAAACAGTGGGCTGTACTCCAGTGCTGGGACATTGA
ATGACTGTATGCTGCTTTTGATTTCCGTTA

The delivery of the therapeutic agents targeting one or more of these miRNAs of interest—miR-424, miR-503, miR503HG, miR-542, miR450a1, miR-450a2, and miR-450b can be based on viral or non-viral systems. Non-viral delivery systems include one or more cationic lipids and cationic polymers that form cationic liposomes. Viral delivery systems include without limitation adeno-associated viral systems and retroviral systems.

Forced Expression of miR-322/-503 Impedes Muscle Growth

Among the H19X microRNAs, miR-322, miR-503, and miR-542 are most frequently associated with muscle atrophy and cachexia. The miR-322/-503 cluster has been correlated with a broad range of diseases and has higher expression levels than miR-542. Disclosed here are therapeutic agents targeting the role of miR-322/-503 in regulating muscle mass.

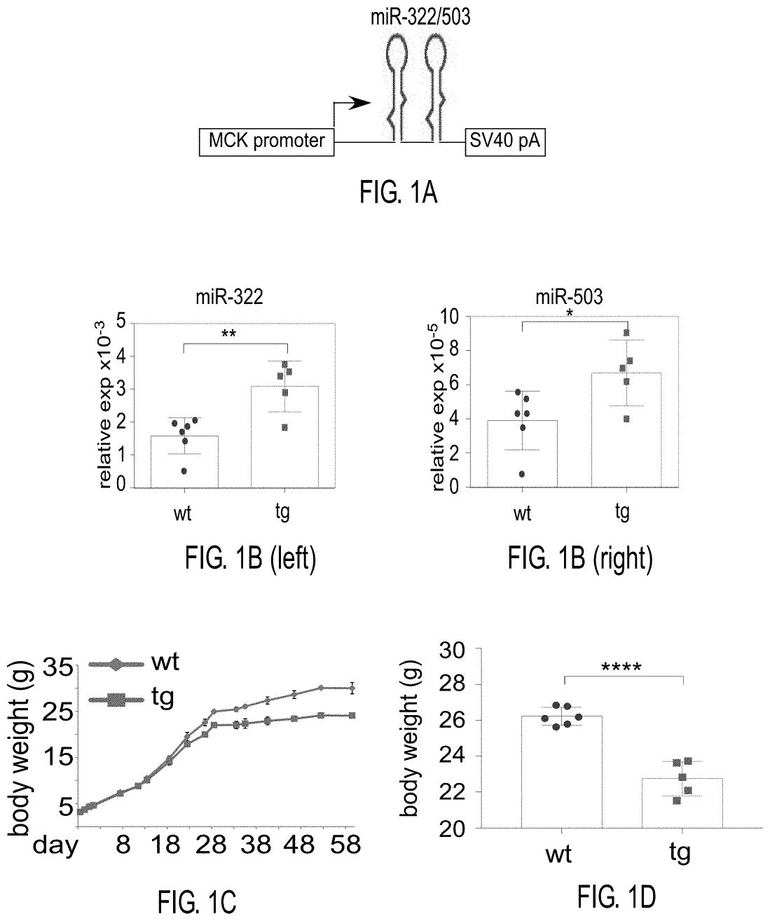

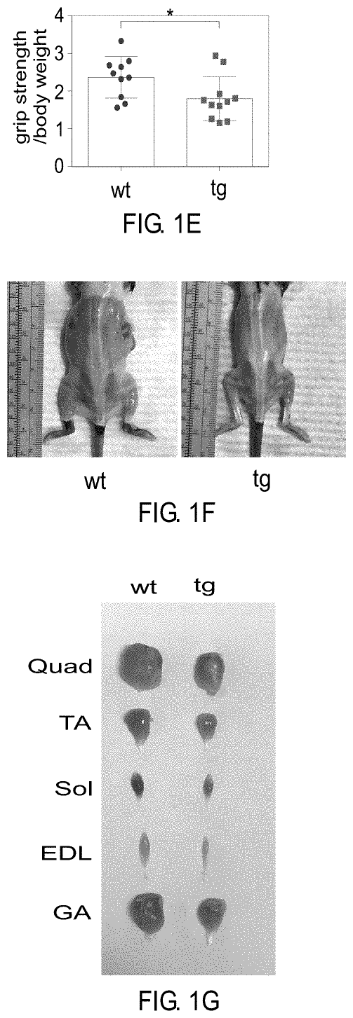

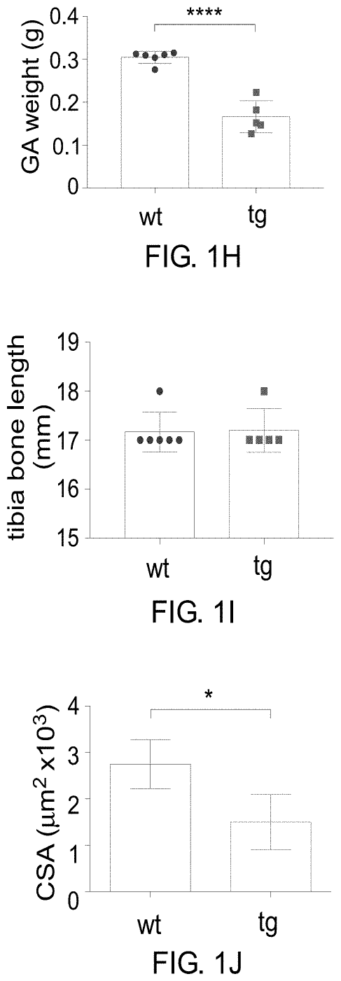

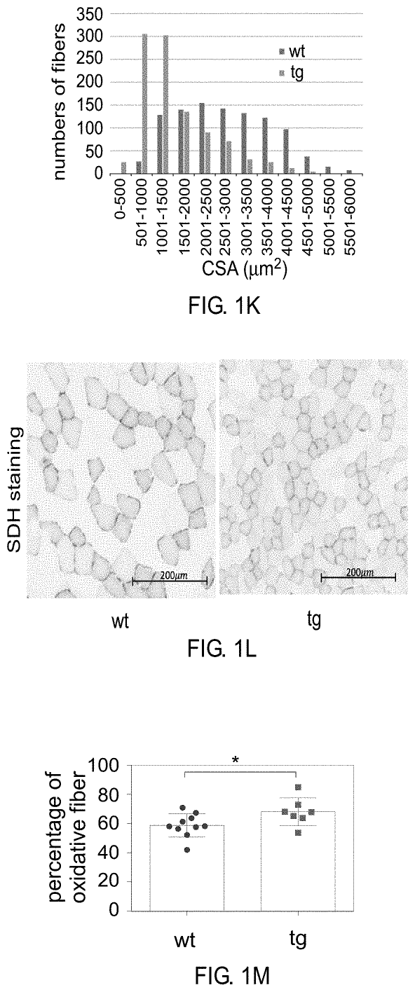

Transgenic mouse strain was generated in which the expression of miR-322/-503 is driven by the MCK promoter (FIG. 1A). FIG. 1A is a diagrammatic representation of the transgenic gene expression cassette. The −6.5 kb MCK promoter was used to drive miR-322/-503 expression. The MCK promoter was chosen because it is highly restricted to striated muscle, and its activity begins on embryonic day 17, which minimizes disturbing the normal function of miR-322/-503 during early embryogenesis. The expression level of the transgene was −2 fold of the endogenous allele at birth. FIG. 1B is a set of graphical representations of the levels of expression of miR-322 (left panel) and miR-503 (right panel) in skeletal muscles of the wild-type and transgenic animals at birth (n=6 vs. 5).

Body masses of transgenic mice became progressively lower than that of wild-type since 4 weeks old. FIG. 1C is a graphical representation of the body weight of the wild-type and transgenic animals. The transgenic animals had lower body weights than non-transgenic littermates starting from 28 days of age. FIG. 1D is a graphical representation of the body weight of the wild-type and transgenic animals at 4 months of age (n=6 vs. 5). The body weights of transgenic animals were significantly lower than non-transgenic littermates at 4 months of age (n=6 vs. 5). At 4-month, transgenic mice weighed 13.2% less than their wild-type littermates (26.22±0.2 g vs. 22.75±0.43 g, wt vs. tg). FIG. 1E is a graphical representation of the grip strength of the wild-type and transgenic animals. Transgenic animals had lower grip strength (n=10 vs. 11). Autopsy revealed widespread muscle atrophy (FIGS. 1F and 1G). FIG. 1F is a set of photographs of the transgenic (right panel) and wild-type (left panel) animals with back and hind limb muscles exposed. FIG. 1G is a photograph of the hind limb muscles from transgenic and wild-type animals. Quad refers to quadriceps, TA refers to tibialis anterior muscles, Sol refers to soleus muscles, EDL refers to extensor digitorum longus muscles, and GA refers to gastrocnemius muscles. The weight of GA muscles of transgenic mice was only 54.5% of the weight of wild-type littermates (0.3048±0.0058 g vs. 0.1662±0.0167 g, wt vs. tg). FIG. 1H is a graphical representation of the masses of GA muscles of the wild-type and transgenic animals. Masses of GA muscles were lower in transgenic animals at 4 months of age (n=6 vs. 5). There was no significant difference in tibia bone length among transgenic and wild-type mice, suggesting that skeletal growth was not affected by the transgene. FIG. 1I is a graphical representation of the tibia bone lengths of the wild-type and transgenic animals. Tibia bone lengths were comparable between transgenic and wild-type animals (n=6 vs. 5).

Histomorphometric analysis of skeletal muscles was performed. FIG. 1J is a graphical representation of the mean cross-sectional area (CSA) of TA muscles of the wild-type and transgenic animals (n=5 vs. 5). FIG. 1K is a graphical representation of the frequency histograms of CSA in TA muscles of the wild-type and transgenic animals (n=5 vs. 5). Results showed that overexpression of miR-322/-503 caused a decrease in average myofiber CSA (FIG. 1J) and a remarkable leftward shift in the myofiber size-frequency histogram (FIG. 1K). Fast-to-slow fiber switching is associated with malnutrition and inflammation, as seen in fasting and glucocorticoid-induced atrophy. miR-322/-503 is one of the top microRNAs induced by glucocorticoids in myotubes. FIG. 1L is a set of photographs of the succinate dehydrogenase (SDH) activity staining of the wild-type and transgenic animals to distinguish between oxidative and low-oxidative muscle fibers. FIG. 1M is a graphical representation of the oxidative fiber percentage based on SDH staining of the wild-type and transgenic animals (n=10 vs. 7). For all panels, *, p<0.05; **, p<0.01; ****, p<0.0001. Consistently, staining for succinate dehydrogenase activity revealed that transgenic muscles had an increase in the percentage of oxidative fibers compared to wild-type littermates (58.7±2.5% vs. 68.1±3.6%, wt vs. tg) (FIG. 1L, 1M). Taken together, these results suggest that overexpression of miR-322/-503 is sufficient to impede skeletal muscle growth in vivo.

### Genetic Inactivation of the H19X Locus Promotes Muscle Growth and Inhibits Muscle Atrophy

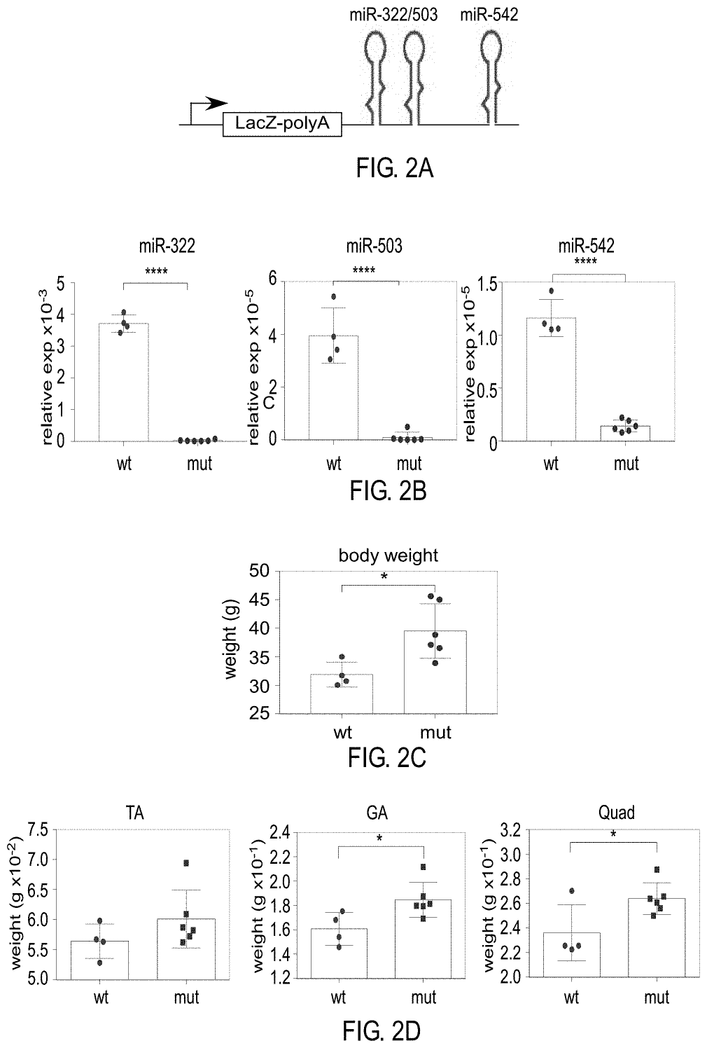

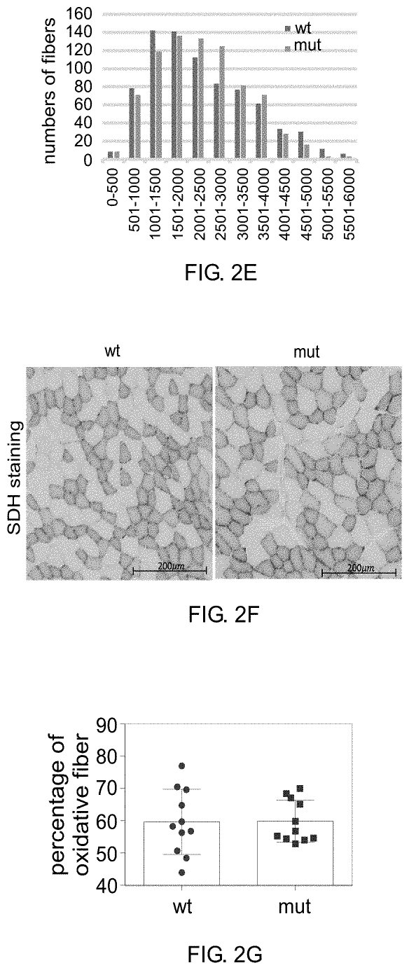

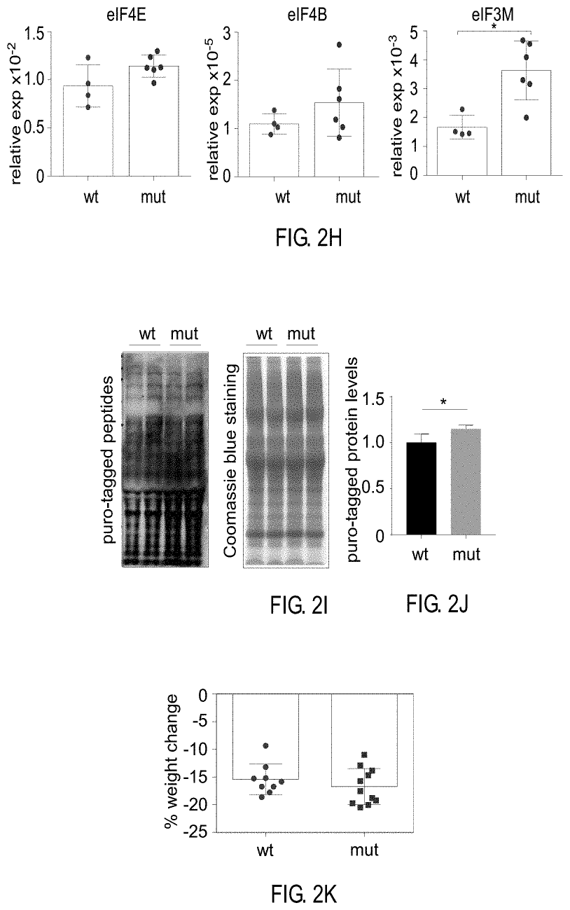

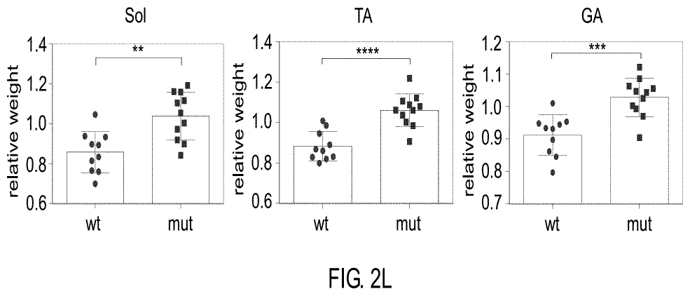

H19X microRNAs were evaluated as negative regulators of skeletal muscle growth by investigating whether their ablation promotes muscle hypertrophy in vivo. The H19X-LacZ mouse strain is defective in transcribing H19X microRNAs, because of an upstream poly-A transcription termination signal (FIG. 2A). FIG. 2A is a diagrammatic representation of the transgenic gene expression cassette with the insertion mutation that blocks downstream transcription in the H19X locus. FIG. 2B is a set of graphical representations of the levels of expression of miR-322 (left panel), miR-503 (middle panel), and miR-542 (right panel) in mutant vs. wild-type muscles (n=4 vs. 6). The expression of miR-322, miR-503, and miR-542 was diminished in skeletal muscles of mutant (i.e., H19X-LacZ) mice (FIG. 2B). Mutant (mt) mice showed overgrowth starting from 4 weeks of age (not shown). FIG. 2C is a graphical representation of the body weight of the wild-type and mutant animals at 6 months old. The bodyweight of mutant mice was significantly higher compared to age-matched wild-type mice (31.88±1.0 g vs. 39.5±1.9 g, wt vs. mt) (FIG. 2C).

Furthermore, FIG. 2D is a set of graphical representations of the hind limb muscle masses of the wild-type and mutant animals, specifically TA (left panel), GA (middle panel), and Quad muscles (right panel). The wet weight of hind limb muscle was higher in mutant mice than in wild-types (n=4 vs. 6), with the gastrocnemius and quadriceps muscle showing significant differences (GA, 0.1608±0.0066 g vs. 0.1847±0.0058 g; Quad, 0.2360±0.0114 g vs. 0.2639±0.0052 g, wt vs. mt) (FIG. 2D).

Morphometric analysis depicted a shift toward thicker myofiber in skeletal muscle of mutant mice. FIG. 2E is a graphical representation of the frequency histograms of CSA in the wild-type and mutant muscles (n=5 vs. 5). FIG. 2F is a set of photographs of the SDH activity staining of the wild-type and mutant animals. FIG. 2G is a graphical representation of the percentage of oxidative fibers in mutant animals and wild-type animals as determined by SDH staining (n=11 vs. 11). There was no significant difference in the percentage of glycolytic and oxidative fibers in skeletal muscle of wild-type and mutant mice, as assayed with SDH staining (FIGS. 2F, G). Altogether, inactivation of the H19X locus causes hypertrophy in both glycolytic and oxidative myofibers.

FIG. 2H is a set of graphical representations of the levels of expression of eIF4E (left panel), eIF4B (middle panel), and eIF3M (right panel) expression in mutant vs. wild-type muscles (n=4 vs. 6). Evaluation of the expression levels of eIFs showed that eIF3M was significantly higher in mutant mice, whereas increases in eIF4E, eIF4G1, eIF4B, and eIF2B5 were not significant (FIG. 2H and data not shown). There appears to be parallel or compensatory mechanisms that also contribute to the regulation of eIF abundance; hence, the effect of loss of H19X microRNAs was partially masked. There was a significant increase in protein synthesis in mutant muscles. FIG. 2I is a set of photographs of the Western Blot analysis of the new protein synthesis in mutant vs. wild-type muscles.

Starvation stimulates the expression of miR-322 and miR-542, two microRNAs that are associated with muscle atrophy and cachexia in human chronic diseases. Inactivating the H19X locus can rescue starvation-induced skeletal muscle atrophy. In H19X mutant and wild-type animals, 48-hour starvation resulted in comparable loss of bodyweight (15.4±0.9% in wt and 16.7±0.9% in mt) (FIG. 2J). Importantly, mutant animals maintained more hind limb muscle. FIG. 2J is a graphical representation of the new protein synthesis in mutant vs. wild-type muscles, which was upregulated in mutant muscles. FIG. 2K is a graphical representation of the body weight loss in mutant vs. wild-type animals after 48 hours of starvation. Bodyweight loss was comparable in mutant and wild-type animals after 48 hours of starvation (n=9 vs. 11). FIG. 2L is a set of graphical representations of the relative weight of sol (left panel), TA (middle panel), and GA (right panel) muscles. Interruption of the H19X locus partially rescued starvation-induced muscle wasting (n=10 vs. 11). Shown are muscle-weight/bodyweight ratios of starved animals normalized to that of regularly fed control littermates.

H19X ncRNAs are Elevated in Skeletal Muscle of the Elderly.

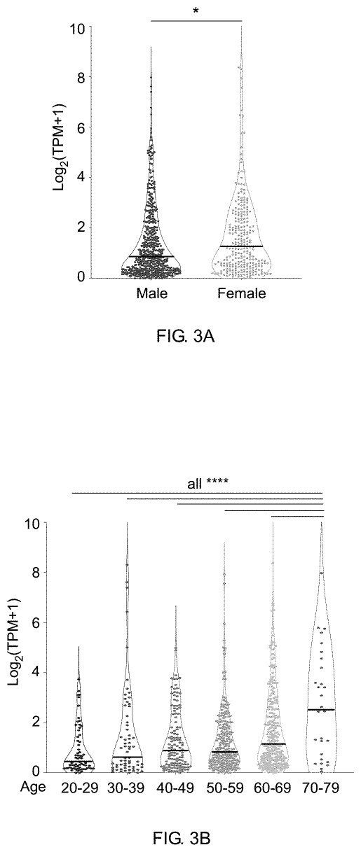

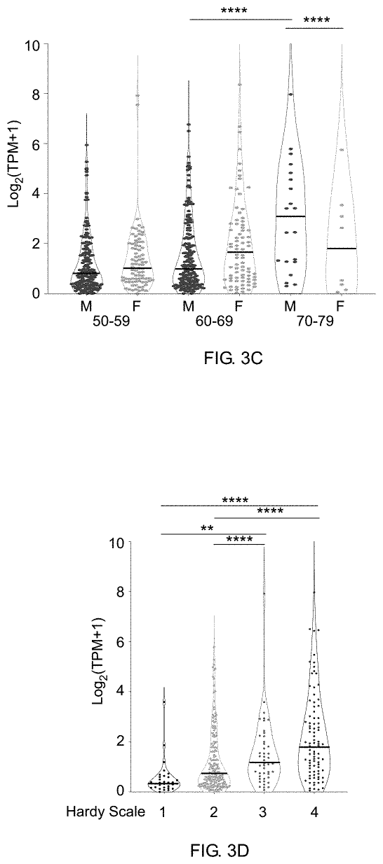

H19X ncRNAs were upregulated in atrophying skeletal muscle in animal models. The levels of H19X ncRNAs were evaluated for expression and regulation in skeletal muscle during aging. The expression of miR503 host gene (miR503HG) in skeletal muscles were analyzed from samples collected in the Genotype-Tissue Expression (GTEx) project. FIG. 3A is a graphical representation of the levels of miR503HG in male and female subjects. Higher levels of miR503HG are associated with female sex. FIG. 3B is a graphical representation of the levels of miR503HG among subjects in ten-year intervals of age, starting from age 20 and up to age 79. FIG. 3C is a graphical representation of the levels of miR503HG in male and female subjects among subjects in ten-year intervals of age, starting from age 50 and up to age 79. In 803 samples (543 Male, 260 Female), the following were observed (a) the expression of miR503HG is markedly higher in females (1.7 folds, Female vs. Male) (FIG. 3A); (b) there is a clear trend that the expression increases with aging, with the expression in age 70-79 group being 15 folds of that in the 20-29 group (FIG. 3B); (c) there is a greater increase between age 70-79 and 60-69 groups in males and 70-79 becomes the only group in which the expression of miR503HG is higher in males than females (FIG. 3C). Higher levels of miR503HG are associated with older age and slow death.

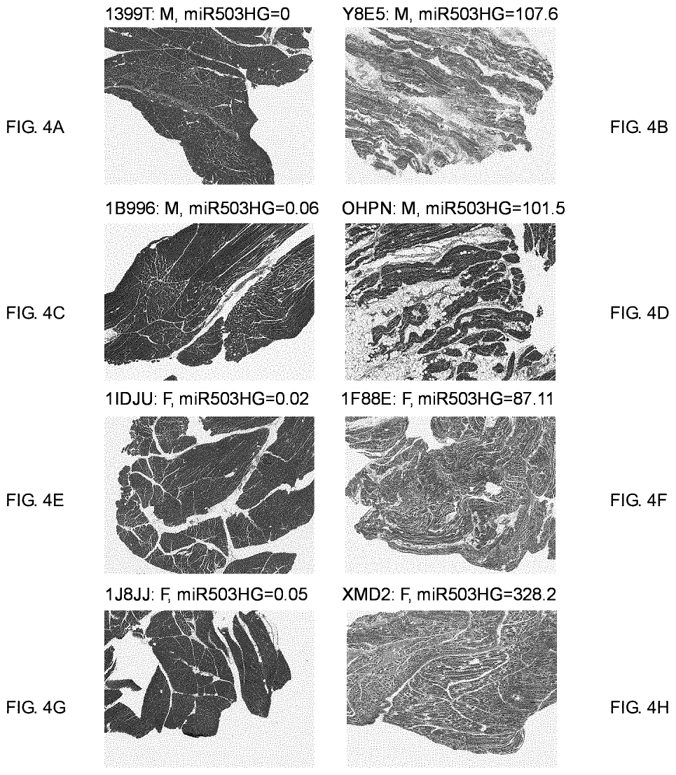

FIGS. 4A-4H are photographic images of levels of miR503HG in male and female subjects at the age of 60-69. High levels of miR503HG are indicative of severe human muscle atrophy. In some individuals of the elder age groups, the expression of miR503HG reaches >1,000 folds of healthy individuals. The muscles of such individuals had moderate to severe atrophy, often accompanied by interfiber fat filtration; men with high levels of miR503HG had more severe atrophy than women (FIGS. 4A-4H). These observations indicate that the levels of H19X ncRNAs are inversely related to muscle mass and H19X ncRNAs are responsible for the gradual loss of muscle mass during aging. The highest miR503HG levels are in the age 70-79 male group (FIG. 3C), because sarcopenia appears to be more prevalent in males than females in elder age groups while this sex difference is not seen in younger age groups.

Higher expression of miR503HG was observed in muscles from individuals who suffered a slow death than those who suffered a quick death. The GTEx project uses the 4-point Hardy Scale to categorize speed of death: 1 for violent and fast death; 2 for fast death of natural causes such as myocardial infarction; 3 for intermediate death such as patients who were ill but death was unexpected; 4 for slow death after a long illness and deaths were not unexpected. FIG. 3D is a graphical representation of the levels of miR503HG at various points on a Hardy scale. Median level is indicated by a horizontal line. *, p<0.05; **, p<0.01; ****, p<0.001. The expression of miR503HG is marked higher in Hardy Scale 3 and 4 groups (underlying illness) than in Hardy Scale 1 and 2 groups (relatively healthy), with the expression in Hardy Scale 4 being 15.5 folds of that in Scale 1 (FIG. 3D). Increased transcription from the H19X locus is associated with muscle abnormalities in chronic diseases.

Tetracycline-Induced Cardiomyocyte-Specific Expression of miR-322/503 Caused Rapid Development of Dilated Cardiomyopathy

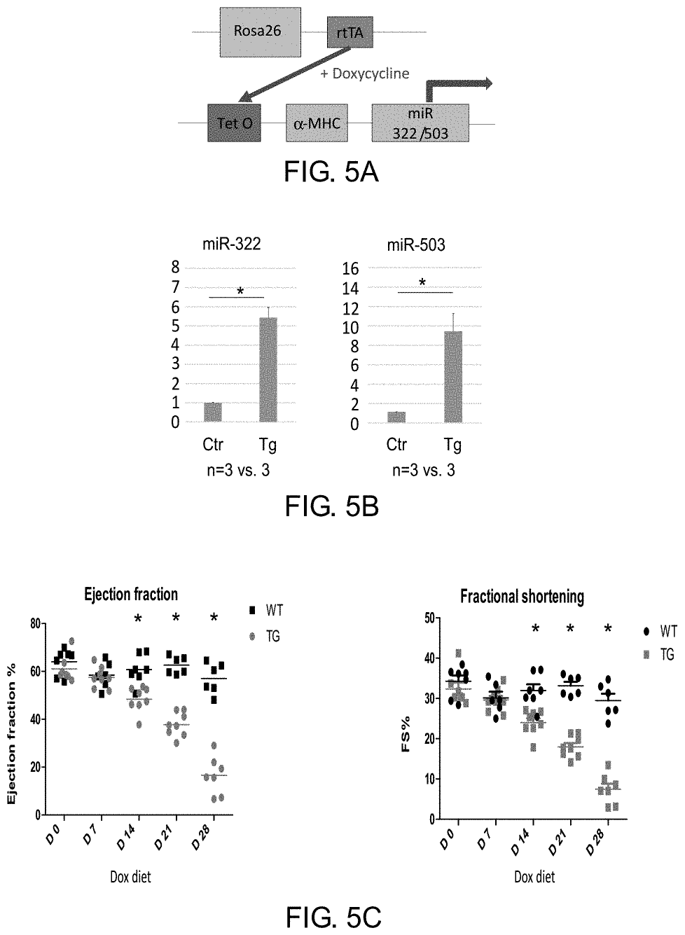

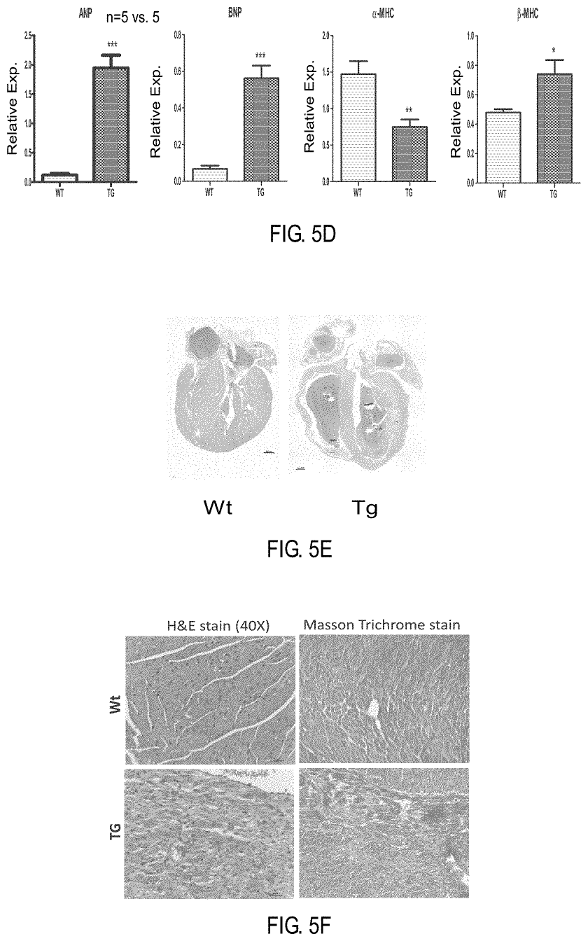

The role of increased miR-322/503 levels in cardiomyopathy were evaluated. An inducible heart-specific transgenic mouse strain was developed, in which ectopic miR-322/503 is triggered by doxycycline (dox) diet. Continuous dox diet induced expression levels comparable to that in embryonic hearts (See FIGS. 5A and 5B as compared to FIG. 3A). FIG. 5A is a diagrammatic representation of the transgene strategy. FIG. 5B is a set of graphical representation of the heart expression of the transgene: miR-322 (left panel) and miR-503 (right panel).

Transgenic animals appeared lethargic as early as 2 weeks after the start of dox diet. No such animals survived beyond 4 weeks (n=8), while transgenic animals (n=10) fed with regular chow were normal and healthy. Assessment of cardiomyopathy molecular markers showed significantly increased expression of ANF and BNP and upregulated β-MHC/α-MHC ratio at 4 weeks. FIG. 5D is a set of graphical representation of the expression of markers of cardiac remodeling: ANF (first panel from the left), BNP (second panel), α-MHC (third panel), and β-MHC (fourth panel). Ectopic miR-322/503 induced markers of cardiac remodeling.

Echocardiography assessment of cardiac function showed progressive deterioration of ejection fraction (EF) and fraction shortening, starting from 2 weeks on dox diet. FIG. 5C is a set of graphical representation of the effect of ectopic miR-322/503 on the systolic pump function of the heart, specifically ejection fraction (left panel) and fractional shortening (right panel). EF was approximately −36% at 3 w. Histological analysis showed enlarged left and right ventricles, and thinning of the ventricular walls. FIG. 5E is a set of photographic images of the heart in wild type and transgenic animals. Dilation of ventricles and thinning of ventricular walls was observed in miR-322/503-overexpressing animals. Cardiomyocytes were disarrayed; and Masson's Trichrome staining showed widespread fibrosis. These results strongly suggest that cardiac specific induction of the expression of miR-322/503 caused dilated cardiomyopathy. FIG. 5F is a set of photographic images of muscles of wild type and transgenic animals stained with hematoxylin and eosin (H&E) stain (top and bottom, left panels) and Masson's Trichrome stain (top and bottom, right panels). Ectopic miR-322/503 induced fibrosis and misaligned cardiomyocytes. H19X non-coding RNAs are responsible for skeletal muscle wasting and heart failure. Embodiments include compositions and methods of targeting these non-coding RNAs in treating these diseases.

Inhibition of miR-322/-503 can Attenuate Starvation-Induced Atrophy in Cultured Myotubes

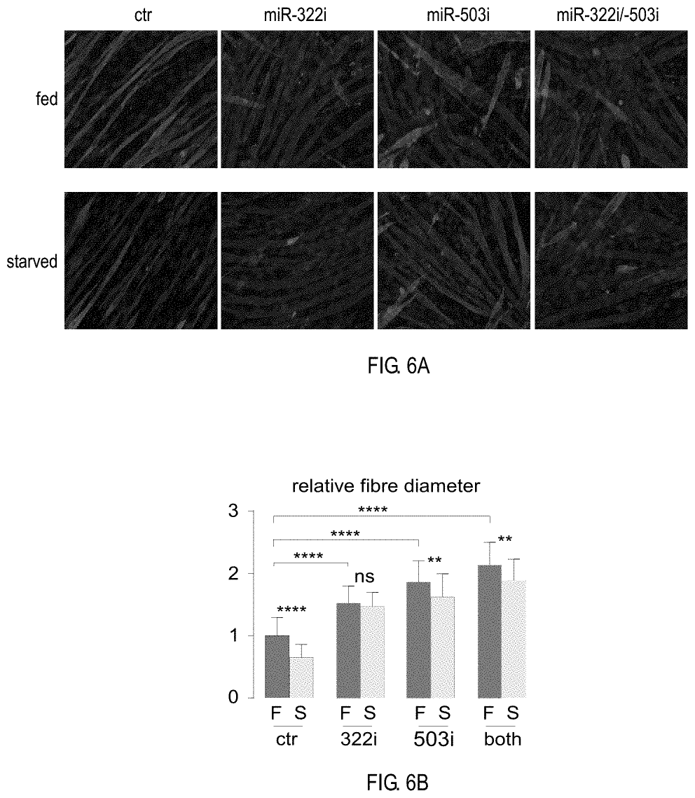

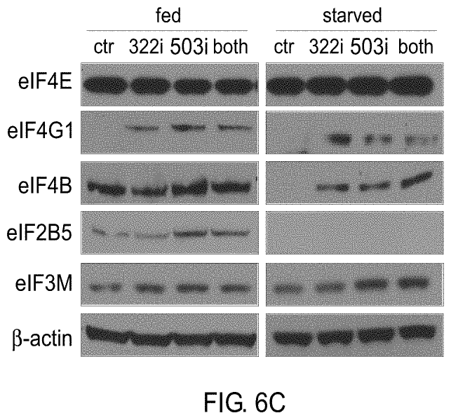

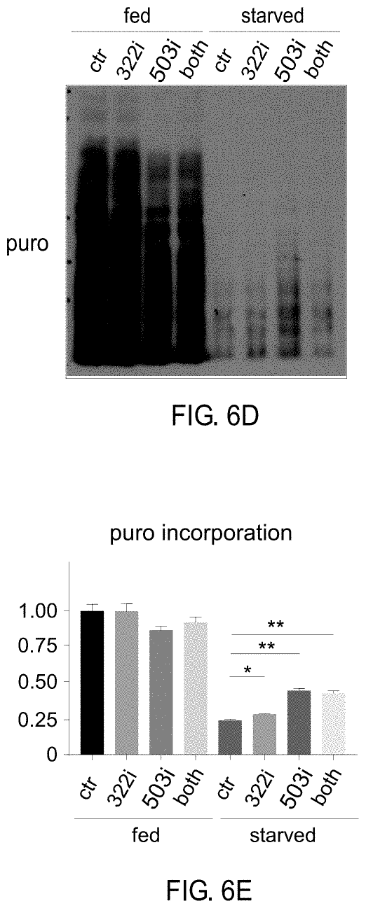

Inhibitors of miR-322/-503 augmented myotube growth and resistance to starvation-induced atrophy. C2C12 myoblasts overexpressing antisense inhibitors of miR-322/-503 (miR-322i/-503i) were differentiated into myotubes and then cultured in regular differentiation media or subjected to starvation in low-serum/low-glucose media for 6 hours. In regular differentiation media, miR-322i/-503i-harboring myotubes had greater diameters, with combined miR-322i and miR-503i showing the strongest effect. Low-serum/low-glucose media induced a marked decrease in myotube diameter in control cultures. Remarkably, miR-322i/-503i partially rescued atrophy in cultured myotubes, with both inhibitors being most effective. FIG. 6A is a set of photographic images of C2C12-derived myotubes treated with control, miR-322i, miR-503i, or both, under normal and starvation conditions. FIG. 6B is a graphical representation of the myotube diameter of C2C12-derived myotubes treated with control, miR-322i, miR-503i, or both, under normal and starvation conditions, showing that both miR-322i and miR-503i upregulated myotube growth and partially protected them from starvation-induced wasting. ns, not significant.

Further, amount of eIF4G1, eIF4B, eIF2B5, and eIF3M proteins was markedly higher in the presence of miR-322i/-503i, either in regular differentiation or starvation culture, or both. FIG. 6C is a photographic image showing expression of eIF4G1, eIF4B, eIF2B5, eIF3M, and eIF4E C2C12-derived myotubes treated with control, miR-322i, miR-503i, or both, under normal and starvation conditions. Expression of eIF4G1, eIF4B, eIF2B5 and eIF3M proteins, but not eIF4E, were boosted by miR-322i/-503i. The presence of miR-322i/-503i also resulted in higher rate of protein synthesis in starved myotubes. FIG. 6D is a photographic image showing expression of puro-tagged peptides in C2C12-derived myotubes treated with control, miR-322i, miR-503i, or both, under normal and starvation conditions. FIG. 6E is a graphical representation of the expression levels of puro-tagged peptides in C2C12-derived myotubes treated with control, miR-322i, miR-503i, or both, under normal and starvation conditions. Impaired protein synthesis in starved myotubes was partially rescued by miR-322i/-503i. For all panels, *, p<0.05; **, p<0.01; ****, p<0.0001. Collectively, these results suggest that miR-322i/-503i promotes growth and attenuates starvation-induced myotube atrophy. The miR-322/-503 is a negative regulator of muscle mass. Inhibitors against miR-322 and miR-503 can preserve muscles in wasting conditions.

Expression of miR-322/miR-503 is Inversely Related to Postnatal Skeletal Muscle Growth.

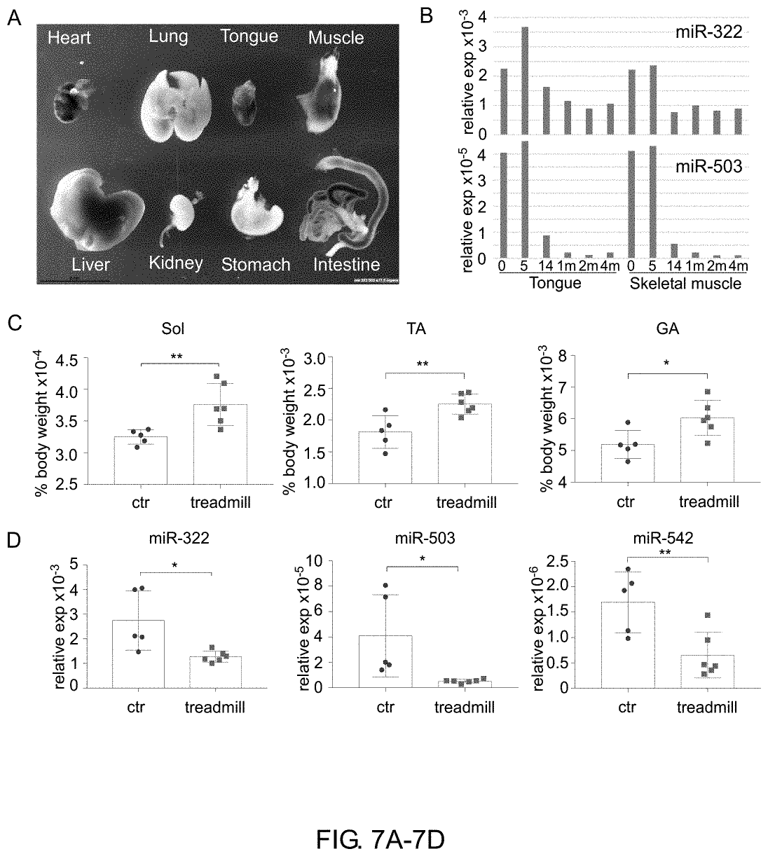

Expression of miR-322(424)/miR-503 is highly enriched in heart and somites in early embryos. Tissue distribution and expression level of H19X miRNAs change in postnatal life stages. FIGS. 7A-7D demonstrate that expression of miR-322/miR-503 is inversely related to postnatal skeletal muscle growth. FIG. 7A is a photographic image of the whole-mount β-gal staining of embryonic organs at E17.5. While the expression specificity in the heart and skeletal muscle is maintained, the levels of miR-322 and miR-503 decreased progressively. FIG. 7B is a graphical representation of the expression levels of miR-322 (upper panel) and miR-503 (lower panel) expression in both tongue and hindlimb muscles. The expression levels are high at birth (0 day) and 5 days in both tongue and hindlimb muscles, and decreased at 14 days and are sustained a low level afterward. To understand the potential role of H19X-encoded miRNAs in regulating muscle mass, their levels are investigated in animal models of muscle hypertrophy. As a model of muscle hypertrophy, the animals were exercised on a rodent treadmill for 4 weeks, which resulted in a significant increase in soleus, TA, and gastrocnemius muscle mass FIG. 7C is a graphical representation of the percentage increase in mass of Sol, TA, and GA muscles as compared to the body weight in control mice and mice subject to four weeks of mandatory treadmill training. Treadmill training led to hypertrophy of hindlimb muscles (n=5 vs. 6). Interestingly, levels of miR-322, miR-503, and miR-542 were markedly lower in TA muscle of exercised mice compared with controls. FIG. 7D is a graphical representation of the expression levels of miR-322 (left panel), miR-503 (middle panel), and miR-542 (right panel) expression in control mice and mice subject to four weeks of mandatory treadmill training. Treadmill training resulted in down-regulation of miR-322, miR-503, and miR-542 in TA muscles (n=5 vs. 6). For all panels, *P<0.05, **P<0.01, and ****, p<0.0001. Muscles have lower levels of H19X non-coding RNAs after exercise. These results demonstrate an inverse relationship between the levels of H19X miRNAs and muscle mass. Elevated levels of miR-322, miR-503, and miR-542 do not support skeletal muscle growth. Therefore, inhibitors of H19X non-coding RNAs would facilitate muscle building in metabolic syndromes.

Among H19X-encoded non-coding RNAs, there are 7 miRNAs of interest—miR-424, miR-503, miR-542, miR450a1, miR-450a2, and miR-450b. Embodiments include antagomirs targeting one or more of these miRNAs. These antagomirs may be delivered as synthetic oligoes or by adeno-associated virus. These miRNAs can also be targeted by RNA-sponge-based technologies. RNA sponges are specially-designed synthetic RNAs that will be targeted by the aforementioned miRNAs. Therefore, these miRNAs will not be able to bind and degrade their natural targets. The RNA-sponges will be delivered as synthetic mRNAs or by adeno-associated virus. H19X-encoded miR503HG is a long non-coding RNA which can be targeted by siRNA-based technologies. Because the entire H19X locus is activated in skeletal muscle wasting, cachexia, sarcopenia and heart failure, the H19X locus can be targeted by gene editing tools such as, but not limited to, integrases (recombinases), clustered regularly interspaced short palindromic repeat (CRISPR) and CRISPR associated protein (Cas) nucleases, TALAN nucleases, zinc finger nucleases, triplex forming oligonucleotides, or combinations thereof. In an example, the H19X locus can be targeted by a CRISPR-based genome editing, including CRISPR-mediated promoter silencing and CRISPR-mediated removal of the genes.

While the specific embodiments of the present disclosure have been described in detail above, those skilled of ordinary skill in the art will appreciate that the specific embodiments are merely specific illustrative embodiments and the scope of the present disclosure is not limited by them. It is to be understood that the substantial scope of the disclosure is defined by the appended claims and their equivalents.

What is claimed is:

1. A method of preventing or treating a muscle disorder in a subject in need thereof, the method comprising administering an inhibitor of expression of one or more of miR-424, miR-503, miR503HG, miR-542, miR450a1, miR-450a2, and miR-450b.

2. The method of claim 1, wherein the inhibitor contains a nucleic acid binding to all or a part of a base sequence of miR-424.

3. The method of claim 1, wherein the inhibitor contains a nucleic acid binding to all or a part of a base sequence of miR-503.

4. The method of claim 1, wherein the inhibitor contains a nucleic acid binding to all or a part of a base sequence of miR503HG.

5. The method of claim 1, wherein the inhibitor contains a nucleic acid binding to all or a part of a base sequence of miR-542.

6. The method of claim 1, wherein the muscle disorder is sarcopenia, cachexia, or cardiomyopathy.

7. A method of preventing or treating a muscle disorder in a subject in need thereof, the method comprising administering an inhibitor of interaction between one or more of miR-424, miR-503, miR503HG, miR-542, miR450a1, miR-450a2, and miR-450b and their natural target.

8. The method of claim 7, wherein the inhibitor contains a nucleic acid binding to all or a part of a base sequence of miR-424.

9. The method of claim 7, wherein the inhibitor contains a nucleic acid binding to all or a part of a base sequence of miR-503.

10. The method of claim 7, wherein the inhibitor contains a nucleic acid binding to all or a part of a base sequence of miR503HG.

11. The method of claim 7, wherein the inhibitor contains a nucleic acid binding to all or a part of a base sequence of miR-542.

12. The method of claim 7, wherein the inhibitor contains a nucleic acid binding to all or a part of a base sequence of miR450a1.

13. The method of claim 7, wherein the inhibitor contains a nucleic acid binding to all or a part of a base sequence of miR-450a2.

14. The method of claim 7, wherein the inhibitor contains a nucleic acid binding to all or a part of a base sequence of miR-450b.

15. The method of claim 7, wherein the muscle disorder is sarcopenia, cachexia, or cardiomyopathy.

16. A method of preventing or treating a muscle disorder in a subject in need thereof, the method comprising inducing a mutation to reduce expression of one or more of miR-424, miR-503, miR503HG, miR-542, miR450a1, miR-450a2, and miR-450b.

17. The method of claim 16, wherein the mutation is an insertion mutation to block transcription of one or more of miR-424, miR-503, miR503HG, miR-542, miR450a1, miR-450a2, and miR-450b.

18. The method of claim 16, wherein the muscle disorder is sarcopenia, cachexia, or cardiomyopathy.

19. The method of claim 16, wherein inducing a mutation comprises editing a sequence of one or more of miR-424, miR-503, miR503HG, miR-542, miR450a1, miR-450a2, and miR-450b by a nuclease.

20. The method of claim 19, wherein the nuclease is one or more of a meganuclease, a zinc-finger nuclease, a transcription activator-like effector nuclease, and a CRISPR/Cas nuclease.
